(19) United States
(12) Patent Application Publication
(10) Pub. No.: US 2008/0009804 A1
(43) Pub. Date: Jan. 10, 2008

(54) Vascular catheter apparatus and method

(75) Inventor: Toni J. Rosetti, Biloxi, MS (US)

Correspondence Address: BRINKS HOFER GILSON & LIONE/CHICAGO/COOK, PO BOX 10395, CHICAGO, IL, 60610, US

(73) Assignee: Cook Incoporated, Bloomington, IN (US)

(21) Appl. No.: 11/823,317

(22) Filed: Jun. 27, 2007

Related U.S. Application Data
(60) Provisional application No. 60/806,850, filed on Jul. 10, 2006.

(51) Int. Cl. A61M 25/01

(52) U.S. Cl. 604/173

(57) ABSTRACT

An apparatus and method for introducing a secondary wire guide into a patient over an indwelling primary wire guide. The apparatus includes a catheter comprising an elongated shaft having proximal and distal end portions and a main lumen extending through a substantial portion thereof. The distal end portion of the catheter further includes a relatively short secondary lumen defined by an inner partition that subdivides the interior volume of the shaft so as to separate the secondary lumen from the main lumen. A pair of ports are disposed in the distal end of the catheter, one port being in communication with the main lumen and the other being in communication with the secondary lumen. A proximal opening is disposed near the proximal end of the catheter and is in communication with the main lumen. A pair of spaced apart side ports extend through the side wall of the catheter shaft at a location intermediate the distal and proximal ends of the catheter, one port being in communication with the main lumen and the other being in communication with the secondary lumen.

### CROSS-REFERENCE TO RELATED APPLICATIONS

This application claims the benefit of U.S. Provisional Application Ser. No. 60/806,850, filed Jul. 10, 2006, entitled “Vascular Catheter Apparatus and Method”, the entire contents of which are incorporated herein by reference.

### TECHNICAL FIELD

The present invention relates to catheters, an in particular vascular catheters and related devices that are used in performing minimally invasive medical procedures. More particularly, the present invention relates to an improved vascular catheter that has a distal end portion that is partitioned to provide a relatively short secondary lumen in addition to a full length main lumen. Various ports, including a pair of ports in the sidewall of the catheter shaft, communicate respectively with the main and secondary lumens. The arrangement of ports and lumens allows the catheter and a secondary wire guide to be introduced into a patient's vessel by tracking the catheter along a first or primary wire guide that has been previously placed within the patient.

### BACKGROUND OF THE INVENTION

Catheters are used to perform minimally invasive medical procedures, such as coronary angioplasty procedure. In a typical balloon angioplasty procedure, a wire guide is inserted into a patient and advanced through the patient's vessels until the distal end of the wire guide is disposed adjacent to the lesion targeted for treatment. A dilation balloon catheter is then advanced over the wire guide until the balloon is disposed adjacent to the lesion. The balloon is then inflated to compress the lesion, thereby improving flow through the vessel.

Many times, the angioplasty procedure further includes a procedure for compressing and/or removing the lesion. For example, the procedure may include the step of deploying a stent within the vessel to further compress the lesion, or may include the step of removing the lesion with an ablation device. However, the introduction of a second catheter device can be time consuming since the initial or previous catheter device has to first be removed from the wire guide before the second catheter can be introduced. Thus, a second wire guide is sometimes introduced into the patient and positioned along side of the first wire guide, whereby the second wire guide can be used to introduce a second catheter device. However, the introduction of a second wire guide can likewise be time consuming. Thus, there is a need for an apparatus and method for quickly introducing a second wire guide along side of a previously placed wire guide.

In addition, sometimes the wire guide that is initially used to gain access to the target region within the patient is not capable of supporting certain types of catheter devices. Thus, the initial wire guide must be removed and replaced with a second (e.g., stronger or stiffer) wire guide. However, and as pointed out above, the introduction of a second wire guide can be time consuming. Thus, there is a need for an apparatus and method for quickly replacing a previously placed wire guide with a second wire guide.

Further complicating the above-described procedures is the use of “short” and/or “long” wire guides. As will be explained in greater detail below, many catheter devices are designed to work with “short” wire guides that are only coupled to the catheter along the distal-most portion of the catheter shaft. The use of “short” wire guides, which typically have a length approximately the same as that of the catheter devices to which they are coupled, have grown in popularity because they are easier to handle and are less likely to get contaminated during the medical procedure. Nevertheless, “long” wire guides are still preferred by many users because of the superior support provided by passing the wire guide through the entire length of the catheter. In any event, many situations arise where it is desirable to replace a long wire guide with a short wire guide, or visa versa. Thus, there is a need for an apparatus and method for quickly introducing a second wire guide along side of a previously placed wire guide, or replacing a first wire guide with a second wire guide, wherein the first and second wire guides may be either “short” or “long” wire guides, or any combination thereof.

The following is a table of U.S. patents and Published patent applications that disclose examples of some of the above-described procedures and devices for performing these procedures, and the content of these patent references is hereby incorporated by reference. The order in which these patent references are listed has no relevance.

| Patent Ref. No. | Title | Date |
|---|---|---|
| 5,234,407 | Method and Device for Exchanging Cardiovascular | Aug. 10, 1993 |
| | Guide Catheter While a Previously Inserted Angioplasty | |
| | Guide Wire Remains in Place | |
| 5,443,457 | Tracking Tip for a Short Lumen Rapid Exchange | Aug. 22, 1995 |
| | Catheter | |
| 6,613,075 | Rapid Exchange Self-Expanding Stent Delivery | Sep. 02, 2003 |
| | Catheter System | |
| 2004/0176793 | Catheter System with Catheter and Guidewire Exchange | Sep. 09, 2004 |
| 2004/0220473 | Vascular Catheter Guide Wire Carrier | Nov. 04, 2004 |
| 2005/0085856 | Locator and Delivery Device and Method of Use | Apr. 21, 2005 |
| 2005/0267408 | Catheter Having First and Second Guidewire Tubes and | Dec. 01, 2005 |
| | Overlapping Stiffening Members | |
| 2005/0277878 | Catheter Shaft Junction Having a Polymeric | Dec. 15, 2005 |
| | Multilayered Sleeve with a Low Processing | |
| | Temperature Outer Layer | |
| 2006/0047266 | Apparatus and Method for Inserting an Intra-Aorta | Mar. 02, 2006 |
| | Catheter through a Delivery Sheath | |
| 2006/0064074 | Rapid Exchange Catheters Having a Sealed Guidewire | Mar. 23, 2006 |
| | Lumen and Methods of Making the Same | |

### BRIEF SUMMARY OF THE INVENTION

The present invention provides an improved apparatus and method for introducing or exchanging wire guides for use in minimally invasive medical procedures, and in particular, for use in coronary angioplasty procedures. The catheter apparatus of the present invention is intended to assist the user during coronary angioplasty when used as a mechanism to introduce or exchange wire guides that are either “short” or “long” in length, or any combination thereof. The apparatus of the present invention may be used to introduce a secondary wire guide along side of a previously placed primary wire guide in order to have two wire guides disposed with the patient's vessel at the same time. The apparatus of the present invention may also be used to replace a previously placed primary wire guide with a secondary wire guide having different properties, for example, to replace a flexible wire guide with a stiffer wire guide.

The catheter of the present invention reduces user/operator time by enabling the operator to “double” wire a vessel with a second wire without needing to remove the primary wire in advance. In addition, the catheter of the present invention reduces user/operator time because the operator does not have to manually “wire” the same vessel twice. And because the amount of time required to manually “wire” a vessel, particularly a vessel with a lesion, is significant, then eliminating the need to perform this maneuver multiple times represents a significant reduction in the time and cost for the overall procedure.

The catheter of the present invention employs an elongated tubular shaft member having a proximal end portion with a hub and a distal end portion or distal tip that can be radiopaque. The catheter shaft comprises a main lumen that extends substantially the entire length thereof. The distal end portion of the catheter shaft further comprises a relatively short secondary lumen that is defined by an inner partition that subdivides the interior volume of the shaft so as to separate the secondary lumen from the main lumen. A pair of ports are disposed in the distal end of the catheter, one port being in communication with the main lumen and the other being in communication with the secondary lumen. A proximal opening is disposed near the proximal end of the catheter shaft and is in communication with the main lumen. A pair of spaced apart side ports extend through the side wall of the catheter shaft at a location intermediate the distal and proximal ends of the catheter, one port being in communication with the main lumen and the other being in communication with the secondary lumen. In one embodiment of the of the present invention, the side ports are disposed near or adjacent to the location where the proximal end of the partition joins the side wall of the catheter shaft. Thus, the catheter of the present invention has at least five ports or openings in communication with the main and secondary lumens. The main lumen, secondary lumen, and each of the at least five ports and openings are configured for the passage of a wire guide therethrough.

The present invention enables the introduction and/or exchange of wire guides of various lengths, including “short” and “long” wire guides. The introduction and/or exchange of wire guides using the present invention is efficient, simple and easy to use, and reduces the time required to perform these procedures. For example, the present invention enables the exchange of a first short wire guide for a second short wire guide without losing access to the vessel in which the wire guide is disposed. Likewise, the present invention enables the introduction of a second wire guide, either short or long in length, into a vessel in which a first wire guide, either short or long in length, has been previously placed to “double” wire the vessel.

Applications of the present invention include the coronary, peripheral and vascular systems, as well and other regions of the anatomy such as the gastro-intestinal system. The exchange of guide wires of various lengths can include: 1) short wire for short wire; 2) short wire for long wire; 3) long wire for short wire; and 4) long wire for long wire.

These and other advantages, as well as the invention itself, will become apparent in the details of construction and operation as more fully described below. Moreover, it should be appreciated that several aspects of the invention can be used with other types of wire guides and catheter devices.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a perspective view of the preferred embodiment of the vascular catheter apparatus of the present invention;

FIG. 2 is a longitudinal sectional view of the apparatus of FIG. 1 taken along line 2-2 of FIG. 1;

FIG. 3 is an end view of the apparatus of FIG. 1 taken along line 3-3 of FIG. 1;

FIG. 4 is a schematic diagram illustrating a first step of an exemplary method of the present invention and showing a guide catheter and primary wire guide being advance through a patient's vessel and towards a target lesion;

FIG. 5 is a schematic diagram illustrating a step of the exemplary method subsequent to the step illustrated in FIG. 4 and showing the primary wire guide being advanced past the lesion;

FIG. 6 is a schematic diagram illustrating a step of the exemplary method subsequent to the step illustrated in FIG. 5 and showing the catheter apparatus of the present invention and secondary wire guide being coupled to the primary wire guide;

FIG. 7 is a schematic diagram illustrating a step of the exemplary method subsequent to the step illustrated in FIG. 6 and showing the catheter apparatus and secondary wire guide being advanced past the lesion; and

FIG. 8 is a schematic diagram illustrating a step of the exemplary method subsequent to the step illustrated in FIG. 7 and showing the primary and secondary wire guides disposed within the patient's vessel and extending past the lesion.

### DETAILED DESCRIPTION OF THE DRAWINGS AND THE PRESENTLY PREFERRED EMBODIMENTS

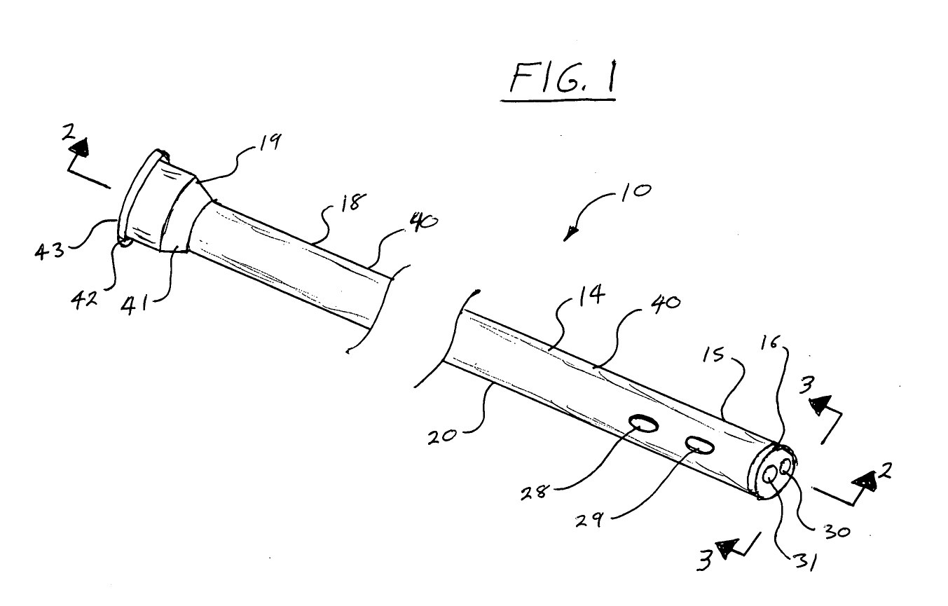

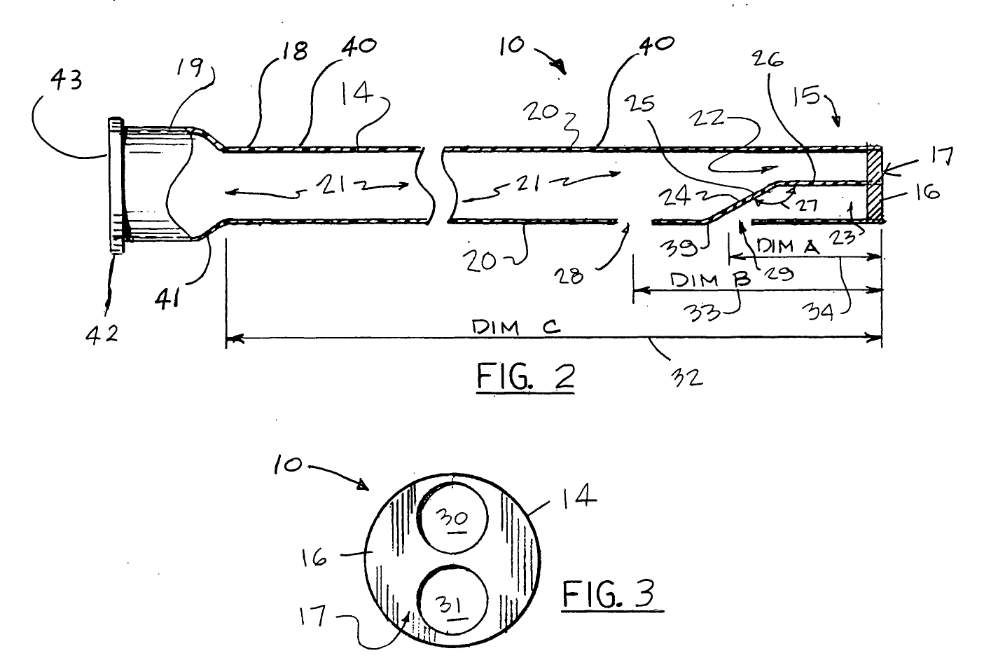

FIGS. 1-3 illustrate a preferred embodiment of the apparatus of the present invention, which is designated generally by the numeral 10. In particular, FIG. 1 is a perspective view of the apparatus 10, FIG. 2 is a longitudinal section sectional view of the apparatus 10 taken along line 2-2 of FIG. 1, and FIG. 3 is an end view of the apparatus 10 taken along line 3-3 of FIG. 1. As will be explained in greater detail below, the apparatus 10 may be utilized to introduce and position a secondary wire guide within a patient's vessel by advancing the apparatus 10 over a primary wire guide that has been previously introduced into the vessel.

The apparatus 10 comprises a vascular catheter apparatus 14. Catheter 14 comprises an elongate shaft 40 having a distal end portion 15 that can be equipped with distal marker 16. The distal marker 16 has a distal surface 17 and generally comprises a radiopaque material. The radiopaque material allows the distal marker 16 to be viewed under fluoroscopy, thereby allowing the position of the distal end portion 15 of the catheter 14 to be determined while disposed within the vessel of the patient. Alternatively, radiopaque markers or materials (not shown) can be embedded in, blended with, or otherwise affixed to the shaft 40 along the distal end portion 15 thereof. Suitable radiopaque markers and materials are well known to those skilled in the art and include high-density metals such gold and materials such as barium sulfate.

Distal marker 16, which is illustrated as covering the distal end portion 15 of the shaft 40, includes two distal openings 30, 31 extending therethrough. In this example the two distal openings are parallel, extend along the longitudinal axis of the shaft 40 and are positioned at the distal end of the catheter. The distal opening 30 communicates with a first distal lumen section 22, and distal opening 31 communicates with a second distal lumen section 23. As best seen in FIG. 2, the first and second distal lumen sections 22, 23 are disposed in a side-by-side arrangement and extend through the distal end portion 15 of the shaft 40. As will be explained in greater detail below, the first and second distal lumen sections 22, 23 are each configured for the passage of a wire guide (not shown) therethrough.

The elongate shaft 40 of catheter 14 has a proximal end portion 18 that includes a proximal hub 19. In the particular embodiment illustrated, the proximal hub 19 has an increased diameter relative to the shaft 40, and is connected to the shaft 40 at its distal end by a flared section 41. The proximal hub 19 further includes a flange 42 at its proximal end, and a proximal opening 43 at its distal end. The proximal opening 43 is in fluid communication with a main lumen 21 of the shaft 40. The proximal hub 19 is preferably configured for attachment to other medical devices. For example, the proximal hub 19 may comprise a female luer fitting that is configured for connection to a medical device having a male luer fitting, such as syringe, which may be used to inject fluids such as saline through the shaft 40 of the catheter 14. In particular, saline is often injected through a vascular catheter to flush air out of the catheter prior to its introduction into the patient.

In the embodiment illustrated, the shaft 40 of catheter 14 comprises a shaft wall 20 that is generally cylindrical in cross-sectional shape. However, other profiles are contemplated, such as an oblong cross-section. Shaft wall 20 surrounds the main lumen 21, which extends through the proximal end portion 18 of the catheter 14. As best seen in FIG. 2, the main lumen 21 is in fluid communication with the first distal lumen section 22, and is similarly configured for the passage of a wire guide therethrough. A partition 24 separates the main lumen 21 and the first distal lumen section 22 from the second distal lumen section 23. Thus, the second distal lumen section 23 is not in fluid communication with either the main lumen 21 or the first distal lumen section 22. The partition 24 contacts wall 20 at position 39, and can include a diagonally extending section 25 and a longitudinally extending section 26. The angle 27 formed by these sections 25, 26 can be an obtuse angle. Alternatively, these sections 25, 26 may be joined by a curved section (not shown), or the entire partition 24 may curved or angled. As will be explained in greater detail below, the partition 24 is shaped and configured to guide a wire guide that is inserted proximally through distal opening 31 and into second distal lumen section 23 towards and out through second side port 29. Likewise, the partition 24 is shaped and configured to guide a wire guide that is inserted distally through first side port 28 and into main lumen 21 towards the first distal lumen section 22 towards and out through distal opening 30.

As mentioned above, a pair of intermediate side ports 28, 29 are provided through the wall 20 of the shaft 40. As best seen in FIG. 2, the first side port 28 is located a relatively short distance proximal of the position 39 where partition 24 contacts wall 20. The second side port is located distally of the position 39, and is either adjacent thereto or spaced a relatively short distance distal to the position 39. Thus, first and second side ports 28, 29 are located on opposite sides of, and are separated by, partition 24. The first side port is in fluid communication with the main lumen 21 and the first distal lumen section 22, and is configured for the passage of a wire guide therethrough. The second side port 29 is in fluid communication with the second distal lumen section 23, and is likewise configured for the passage of a wire guide therethrough. In the embodiment illustrated, the first and second side ports 28, 29 are shown disposed along the same side of the shaft 40. However, these ports 28, 29 may circumferentially disposed anywhere about the circumference of the shaft 40, for example, on opposite sides of the shaft 40.

FIG. 2 further illustrates the length of catheter 14 and the relative positions of first and second side ports. In particular, Dimension C (identified as element 32 in the figure) represents the overall length of catheter shaft 40, as measured from the distal surface 17 of distal marker 16. Dimension B (identified as element 33 in the figure) represents the position of first side port 28, as measured from the distal surface 17 of distal marker 16. Dimension A (identified as element 34 in the figure) represents the position of second side port 29 as measured from the distal surface 17 of distal marker 16.

With respect to the embodiment illustrated, the shaft 40 of the catheter 14 may have an overall length 32 (Dimension C) in the range of 10 cm to 200 cm, and preferably may have a length of about 135 cm. First side port 28 of catheter 14 may be located a distance 33 (Dimension B) in the range of about 1 cm to at least 60 cm from the distal end of the catheter 14, and is preferably located about 20 cm from the distal end of the catheter 14. Second side port 29 of catheter 14 may be located a distance 34 (Dimension A) in the range of about 1 cm to at least 20 cm from the distal end of the catheter 14, and is preferably located about 10 cm from the distal end of the catheter 14.

The catheter 14 may be manufactured or formed from any number of suitable materials. For example, the shaft 40 can be formed by extrusion from PTFE or similar materials. A hydrophilic coating may be applied to the exterior surface of the shaft 40 to enhance the catheter's ability to be advanced through the vessel of the patient. In addition, the stiffness and pushability of the catheter 14 may be enhanced by the addition of a stiffening wire or mandrel (not shown), which may either be embedded in the wall 20 of the shaft 40, or disposed through the interior of main lumen 21. Alternatively, all or a portion of the shaft 40 may comprise a metal hypo tube to provide the catheter 14 with enhanced stiffness and pushability.

The outer diameter of the shaft 40 along the proximal end portion 18 can be about 1 FR to 8 FR, and preferably may be about 3 FR. The outer diameter of the shaft 40 along the distal end portion 15 can similarly be about 1 FR to 8 FR, and preferably may be about 2.3 FR. Thus, the shaft 40 of the catheter 14 may have a stepped outer diameter wherein the distal end portion 15 has a smaller diameter than that of the proximal end portion 18. A stepped a configuration has several advantages over a non-stepped configuration. First, the relatively smaller diameter of the distal end portion 15 facilitates advancement through the vessels of a patient because it provides the distal end of the catheter 14 with a relatively small entry profile. Second, the relatively smaller diameter of the distal end portion 15 provides this portion of the shaft 40 with increased flexibility (relative to the proximal end portion 18 of the catheter 14), which facilitates advancement of the catheter 14 through tortuous vessel pathways. The relatively larger diameter of the proximal end portion 18, on the other hand, provides this portion of the shaft 40 with greater stiffness, which facilitates pushing of the catheter into the patient. Irrespective of the above description, it should nevertheless be understood that a shaft 40 having a constant diameter long the length thereof, or having a larger diameter distal end portion 15 relative to the proximal end portion 18, could also be utilized.

As mentioned above, main lumen 21, first distal lumen section 22, second distal lumen section 23, and the ports and openings in fluid communication therewith (i.e., first and second side ports 28, 29, distal openings 30, 31, and proximal port 43), are each configured for the passage of a wire guide therethrough. Wire guide sizes (diameters) that could be used with the apparatus 10 of the present invention include, as examples, 0.014″, 0.018″, 0.035″ and 0.038″. Thus, the various lumens and ports/openings of catheter 14 should be sized large enough to accommodate the size (or range of sizes) of the wire guides intended to be used therewith.

In addition, and as will be explained in greater detail below, the apparatus 10 of the present invention is configured for use with wire guides of various lengths. As used herein, the term “short” wire guide is used to describe a wire guide having a length that is about equal to (or shorter than) the overall length 32 of the catheter 14, and the term “long” wire guide is used to describe a wire guide having a length that is substantially longer than the overall length 32 of the catheter 14. For example, a typical long wire guide may have a length that is twice as long as the overall length 32 of the catheter 14. In many minimally invasive medical procedures, users often prefer the use of a short wire guide since it is easier to manipulate and less likely to become contaminated or interfere with other aspects of the procedure. However, it may be difficult to exchange catheter devices over a short wire guide since a substantial portion of the wire guide may be disposed within the catheter device, thereby making it difficult to maintain control of the wire guide during the exchange. Thus, the apparatus 10 of the claimed invention is configured to facilitate the introduction and/or exchange of any combination of short and long wire guides.

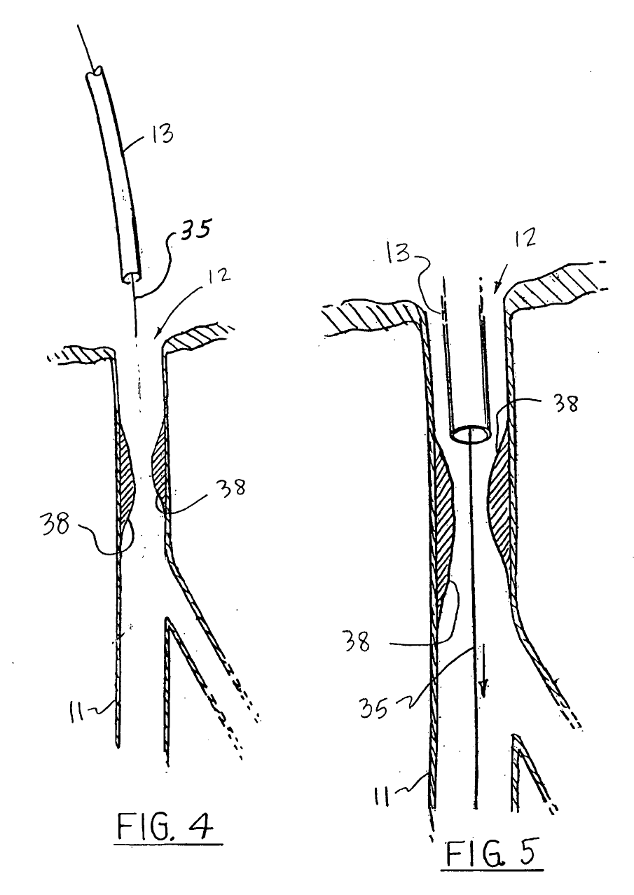

An exemplary method of the present invention will now be described in connection with FIGS. 4-8, which illustrate successive steps for placement of two wire guides through a lesion in a vessel of a patient. FIG. 4 is a schematic diagram illustrating a first step of the exemplary method, and illustrates a guide catheter 13 and primary wire guide 35 being advance through a patient's vessel I land towards a target lesion 38. In particular, the primary wire guide 35 is first disposed through the lumen of the guide catheter 13 while outside the patient, and then the guide catheter 13 and primary wire guide 35 are simultaneously introduced into and advanced through the vessel 11 of the patient until the distal ends of these devices are positioned near the ostium 12 of the vessel 11, for example, near ostium of the coronary artery. Guide catheter 13 provides support to the primary wire guide 35 as these devices are advanced through the patient. As is understood by those skilled in the art, the proximal ends (not shown) of the guide catheter 13 and primary wire guide 35 remain outside the patient during the medical procedure. In an alternative to the above described step, the guide catheter 13 may be first introduced and advanced through the patient, and then the primary wire guide 35 may be subsequently advanced through the guide catheter 13. In yet another alternative, the primary wire guide 35 may be introduced and advanced through the patient without the use of a guide catheter 13.

FIG. 5 is a schematic diagram illustrating a step subsequent to the step illustrated in FIG. 4, and shows the primary wire guide 35 being advanced distally beyond the distal end of the guide catheter 13 and past the lesion 38. The primary wire guide 35 may then be utilized to advance and deliver other catheter devices to the area of the lesion 38 to perform various diagnostic or treatment procedures. For example, the primary wire guide 35 may be used to advance a dilation balloon catheter to the target site (i.e., the lesion 38) within the vessel 11 and perform an angioplasty procedure. Similarly, the primary wire guide 35 may be used to deliver a stent delivery catheter to the target site. The guide catheter 13 may be removed prior to the introduction and advancement of these other medical devices, or may be left in place within the patient (as illustrated in FIG. 5).

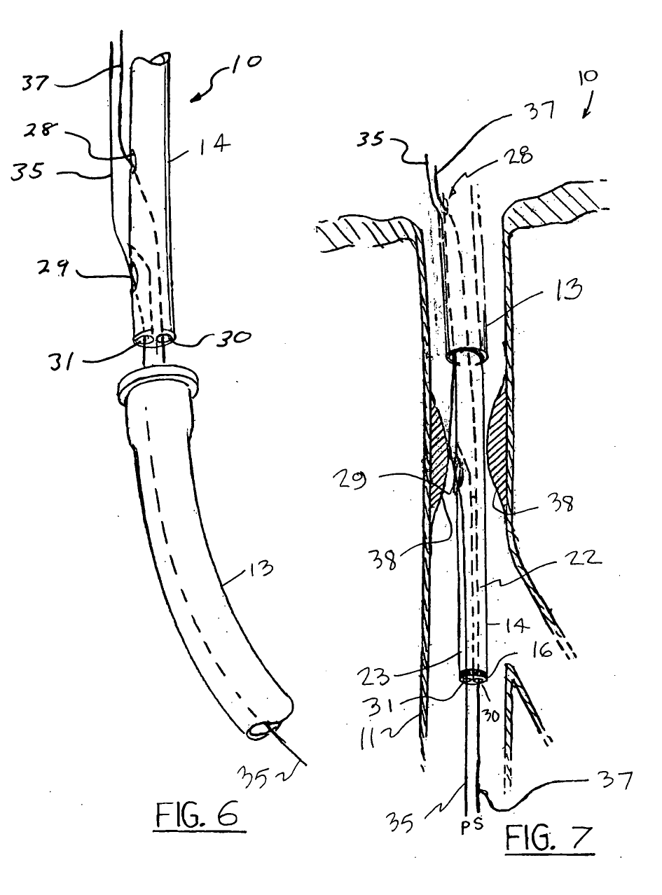

FIG. 6 is a schematic diagram illustrating a step subsequent to the step illustrated in FIG. 5, and shows the catheter apparatus 10 of the present invention being coupled to the primary wire guide 35. In particular, the catheter 14 is coupled to the primary wire guide 35 by inserting the proximal end of the primary wire guide 35 in through distal opening 31, through second distal lumen section 23 (see FIG. 2), and out through second side port 29. At the same time, a secondary wire guide 37 is coupled to the catheter 14 by advancing the distal end of the secondary wire guide 37 in through first side port 28, through main lumen 21 and first distal lumen section 22 (see FIG. 2), and out through distal opening 30. The catheter 14, with the secondary wire guide 37 coupled thereto, is now ready to be advanced along the primary wire guide 35 and into the patient. In the particular embodiment illustrated, both the primary wire guide 35 and the secondary wire guide 37 are short wire guides. However, it should be understood that either or both of these wire guides 35, 37 could be long wire guides.

FIG. 7 is a schematic diagram illustrating a step subsequent to the step illustrated in FIG. 6, and shows the catheter 14 and secondary wire guide 37 being advanced through the vessel 11 and past the lesion 38. This is accomplished by grasping the catheter 14 and the secondary wire guide 37 together, and then pushing both of these elongate members 14, 37 simultaneously along the primary wire guide 35 until the distal ends thereof reached the desired location within the patient's vessel 11. In other words, the primary wire 35 functions as a rail to guide the catheter body 14 into and through the guide catheter 13, down the vessel 11 and beyond the lesion 38. In the particular embodiment illustrated, catheter 14 and the secondary wire guide 37 are shown being advanced through the guide catheter 13. However, it should be understood that the guide catheter 13 is not necessary to the introduction and advancement of the catheter 14 and the secondary wire guide 37 along the primary wire guide 35, and may be eliminated from the procedure.

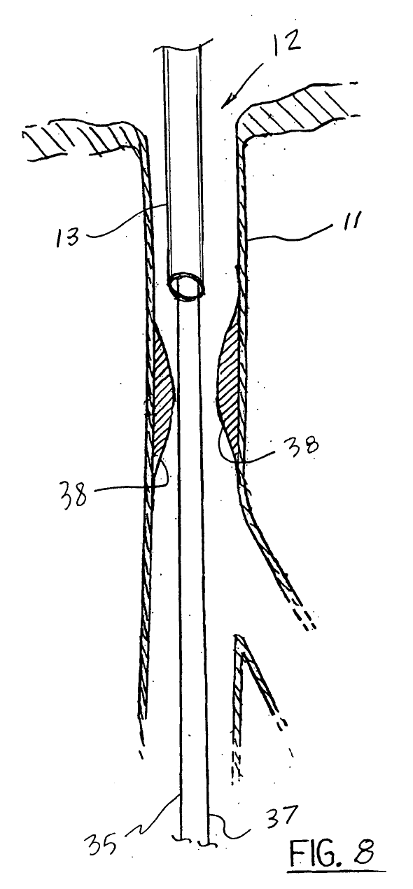

FIG. 8 is a schematic diagram illustrating a step subsequent to the step illustrated in FIG. 7, and shows the primary and secondary wire guides 35, 37 simultaneously disposed within the patient's vessel 11 and extending past the lesion 38. This is often referred to as “double wiring” the vessel. In this step, the catheter 14 has been removed by withdrawing the catheter 14 in a proximal direction until the distal end portion 15 of the catheter 14 decouples from the proximal ends of the wire guides 35, 37. Once the catheter 14 has been removed, either or both of the wire guides 35, 37 may be used to introduce other catheter devices to the target site within the vessel 11. For example, the primary and secondary wire guides 35, 37 may be used to simultaneously introduce to two separate devices, such as a dilation balloon catheter and a stent delivery catheter. This allows the stent to be positioned and deployed within the legion almost immediately after the lesion had been dilated with the balloon, as opposed to having to first remove and then replace the dilation balloon with a stent delivery catheter when using only a single wire guide.

In an alternative to the method step illustrated in FIG. 8, the primary wire guide 35 may be withdrawn and removed at the same time catheter 14 is withdrawn and removed. If so, then only the secondary wire guide 37 would remain within the patient's vessel II for use in introducing further medical devices. Such a procedure is often employed when it is desired to replace a primary wire guide 35 with a secondary wire guide 37 having a different size or stiffness. For example, many procedures are initiated with a relatively small and flexible primary wire guide 35 that is capable of navigating a tortuous pathway through the patient. However, a primary wire guide 35 of this type may not be capable of supporting the introduction and advancement of a relatively large or stiff catheter device there along. Thus, it may be desirable to exchange the primary wire guide 35 for a larger and/or stiffer secondary wire guide 37. If so, then the above-described method can be used to replace the primary wire guide 35 with the secondary wire guide 37. Such a procedure is typically called a wire guide exchange.

In the above-described methods, the apparatus of the present invention is used to introduce secondary wire guide 37 by coupling the secondary wire guide 37 to only the distal end portion 15 of the catheter, i.e., by extending the secondary wire guide 37 through first side port 28, through main lumen 21 and first distal lumen section 22 (see FIG. 2), and out through distal opening 30. This arrangement is referred to as a short wire or rapid exchange configuration. However, the secondary wire guide 37 may alternatively be coupled to the catheter 14 by extending it through proximal opening 43 (as opposed to first side port 28), through main lumen 21 and first distal lumen section 22 (see FIG. 2), and out through distal opening 30. In other words, the secondary wire guide 37 extends through substantially the entire length of the shaft 40. This arrangement is referred to as a long wire or over-the-wire configuration. Some users may prefer a long wire coupling configuration because the secondary wire guide 37 remains within, and is therefore supported by, the catheter 14 over the entire length of the catheter 14. However, it should be appreciated that a long wire coupling configuration requires a much longer (i.e., “long”) secondary wire guide 37 to facilitate removal of the catheter 14 after the secondary wire guide 37 has been introduced into the patient. More specifically, the proximal portion of the secondary wire guide 37 extending out of the patient (after introduction) must be longer than the total length of the catheter 14 so that the user may grasp a portion of the exposed secondary wire guide 37 and hold it steady as the catheter 14 is withdrawn out from the patient and proximally over the proximal portion thereof.

The use of the long wire coupling configuration also permits the introduction of the secondary wire guide 37 into the patient to be delayed until after the catheter 14 has been introduced into the patient and advanced to the target region (e.g., lesion 38). More specifically, the method steps described above in connection with FIGS. 6 and 7 could alternatively be performed in the following sequence: a) couple the catheter 14 to primary wire guide 35 by inserting the proximal end of the primary wire guide 35 in through distal opening 31, through second distal lumen section 23, and out through second side port 29; b) advancing the catheter 14 along the primary wire guide 35 to the target region; c) inserting the secondary wire guide 37 through proximal opening 43; and d) advancing the secondary wire guide 37 through main lumen 21 and first distal lumen section 22, and out through distal opening 30 until it reaches the target region within the patient. This alternative method eliminates the need to simultaneously advance the secondary wire guide 37 and the catheter 14 into the patient and long primary wire guide 35, and is less likely to result in accidental or premature uncoupling of the secondary wire guide 37 from the catheter 14.

All measurements disclosed herein are at standard temperature and pressure, at sea level on Earth, unless indicated otherwise. All materials used or intended to be used in a human being are biocompatible, unless indicated otherwise.

It will of course be well understood from the discussions above that the apparatus of the present invention may be used in other medical procedures, and may be used to access other regions of the patient's anatomy. For example, the apparatus may be used to introduce or exchange wire guides during minimally invasive procedures in the patient's gastro-intestinal system, such as procedures for the removal of stones from the common bile duct. As a consequence, the apparatus may include modifications specific to these procedures, such as a longer overall length. It is therefore intended that the foregoing detailed description be regarded as illustrative rather than limiting, and that it be understood that it is the following claims, including all equivalents, that are intended to define the spirit and scope of this invention.

1. A catheter apparatus comprising:
a) an elongated catheter shaft having a proximal end portion, a distal end portion, and a shaft wall surrounding a main lumen, the main lumen extending between the proximal and distal end portions;
b) the distal end portion comprising a secondary lumen defined by an inner partition that subdivides an interior volume of the shaft so as to separate the secondary lumen form the main lumen, the secondary lumen having a length that is shorter than that of the main lumen;
c) a pair of distal ports in the distal end portion of the shaft, one distal port being in communication with the main lumen and the other being in communication with the secondary lumen;
d) a proximal opening in the proximal end portion of the shaft, the proximal opening being in communication with the main lumen; and
e) a pair of spaced apart side ports that extend through the shaft wall at a location intermediate the pair of distal ports and the proximal opening, one side port being in communication with the main lumen and the other being in communication with the secondary lumen,
wherein each of the main lumen, the secondary lumen, the pair of distal ports, the proximal opening, and the pair of side ports are configured for the passage of a wire guide therethrough.

2. The catheter apparatus of claim 1 wherein at least a portion of the inner partition is disposed at an acute angle relative to a longitudinal axis of the catheter shaft.

3. The catheter apparatus of claim 1 wherein the inner partition comprises a distal portion and a proximal portion, the distal portion being disposed parallel to a longitudinal axis of the catheter shaft and the proximal portion being disposed at an acute angle relative to the longitudinal axis.

4. The catheter apparatus of claim 3 wherein the juncture of the distal and proximal portions of the inner partition form an obtuse angle.

5. The catheter apparatus of claim 1 wherein the inner partition comprises a distal end and a proximal end, the distal end engaging a distal end of the catheter shaft, and the proximal end engaging the shaft wall at a location between the pair of side ports.

6. The catheter apparatus of claim 5 wherein the side port that is in communication with the secondary lumen is disposed distal of and adjacent to the location where the proximal end of the inner partition engages the shaft wall.

7. The catheter apparatus of claim 5 wherein the side port that is in communication with the main lumen is disposed proximal of and adjacent to the location where the proximal end of the inner partition engages the shaft wall.

8. The catheter apparatus of claim 1 wherein the side port that is in communication with the main lumen is located a relatively long distance from the proximal opening and a relatively short distance from the distal port in communication with the main lumen.

9. The catheter apparatus of claim 1 wherein the catheter shaft comprises a distal end that is radiopaque.

10. The catheter apparatus of claim 9 wherein the pair of distal ports each extend through the radiopaque distal end of the catheter shaft.

11. The catheter apparatus of claim 1 further comprising a first wire guide, the first wire guide being disposed through one of the main lumen and the secondary lumen.

12. The catheter apparatus of claim 11 further comprising a second wire guide, the second wire guide being disposed through the other of the main lumen and the secondary lumen.

13. The catheter apparatus of claim 1 further comprising a wire guide disposed through the main lumen, wherein the wire guide extends through the side port that is in communication with the main lumen and the distal port that is in communication with the main lumen.

14. The catheter apparatus of claim 1 further comprising a wire guide disposed through the main lumen, wherein the wire guide extends through the proximal opening and the distal port that is in communication with the main lumen.

15. A system comprising:
a) a catheter apparatus comprising an elongated catheter shaft having a proximal end portion, a distal end portion, and a shaft wall surrounding a main lumen, the main lumen extending between the proximal and distal end portions;
i) the distal end portion comprising a secondary lumen defined by an inner partition that subdivides an interior volume of the shaft so as to separate the secondary lumen form the main lumen, the secondary lumen having a length that is shorter than that of the main lumen;
ii) a pair of distal ports in the distal end portion of the shaft, one distal port being in communication with the main lumen and the other being in communication with the secondary lumen;
iii) a proximal opening in the proximal end portion of the shaft, the proximal opening being in communication with the main lumen; and
iv) a pair of spaced apart side ports that extend through the shaft wall at location intermediate the pair of distal ports and the proximal opening, one side port being in communication with the main lumen and the other being in communication with the secondary lumen;
b) a first wire guide disposed through the main lumen of the catheter apparatus; and
c) a second wire guide disposed through the secondary lumen of the catheter apparatus.

16. The system of claim 15 wherein the first wire guide and the second wire guide each have a length that is substantially longer than that of the catheter apparatus.

17. The system of claim 15 wherein the first wire guide and the second wire guide each have a length that is about equal to or shorter than that of the catheter apparatus.

18. The system of claim 15 wherein one of the first wire guide and the second wire guide has a length that is substantially longer than that of the catheter apparatus, and the other of the first wire guide and the second guide has a length that is about equal to or shorter than that of the catheter apparatus.

19. The system of claim 15 further comprising a guide catheter having a lumen disposed therethrough, wherein the catheter apparatus, the first wire guide and the second wire guide are each disposed through the lumen of the guide catheter.

20. A method of placing a second wire guide into a vessel of a patient, comprising the steps of:
a) introducing a primary wire guide into the vessel of the patient and positioning a distal end of the primary wire guide at a target region within the vessel;
b) coupling the primary wire guide to a catheter apparatus, the catheter apparatus comprising an elongated shaft having a first lumen and a second lumen, the first lumen extending between a proximal opening near a proximal end of the shaft and a first distal opening in a distal end of the shaft, and further including a first side port through a wall of the shaft in communication with the first lumen, the second lumen being substantially shorter than the first lumen and extending between a second distal opening in the distal end of the shaft and a second side port through the wall of the shaft, the primary wire guide being coupled to the catheter apparatus by inserting a proximal end of the primary wire guide through the second lumen of the catheter apparatus;
c) coupling a secondary wire guide to the catheter apparatus by extending the secondary wire guide through the first lumen of the catheter apparatus;
d) simultaneously advancing the catheter apparatus and the secondary wire guide in a distal direction and into the vessel by pushing the catheter apparatus along the primary wire guide; and
e) removing the catheter apparatus from the vessel by withdrawing the catheter apparatus in a proximal direction.

21. The method of claim 20 wherein, in step c), the secondary wire guide is coupled to the catheter apparatus by passing the secondary wire guide through the first distal opening and the first side port.

22. The method of claim 20 wherein, in step c), the secondary wire guide is coupled to the catheter apparatus by passing the secondary wire guide through the first distal opening and the proximal opening.

23. The method of claim 20 further comprising the step of removing the primary wire guide from the vessel by withdrawing the primary wire guide in a proximal direction

24. A method of placing a second wire guide into a vessel of a patient, comprising the steps of:
a) introducing a primary wire guide into the vessel of the patient and positioning a distal end of the primary wire guide at a target region within the vessel;
b) coupling the primary wire guide to a catheter apparatus, the catheter apparatus comprising an elongated shaft having a first lumen and a second lumen, the first lumen extending between a proximal opening near a proximal end of the shaft and a first distal opening in a distal end of the shaft, and further including a first side port through a wall of the shaft in communication with the first lumen, the second lumen being substantially shorter than the first lumen and extending between a second distal opening in the distal end of the shaft and a second side port through the wall of the shaft, the primary wire guide being coupled to the catheter apparatus by inserting a proximal end of the primary wire guide through the second lumen of the catheter apparatus;
c) advancing the catheter apparatus in a distal direction and into the vessel by pushing the catheter apparatus along the primary wire guide;
d) coupling a secondary wire guide to the catheter apparatus by advancing a distal end of the secondary wire guide through the proximal opening, through the first lumen and through the first distal opening of the catheter apparatus until the distal end of the secondary wire guide is positioned at the target region within the vessel;
e) removing the catheter apparatus from the vessel by withdrawing the catheter apparatus in a proximal direction.

25. The method of claim 24 further comprising the step of removing the primary wire guide from the vessel by withdrawing the primary wire guide in a proximal direction.
